(19) United States
(12) Patent Application Publication
(10) Pub. No.: US 2024/0190814 A1
(43) Pub. Date: Jun. 13, 2024

(54) Novel Intermediates

(71) Applicant: Syngenta Crop Protection AG, Basel (CH)

(72) Inventors: George Robert Hodges, Bracknell (GB); Martin Charles Bowden, Bracknell (GB)

(73) Assignee: Syngenta Crop Protection AG, Basel (CH)

(21) Appl. No.: 18/551,292

(22) PCT Filed: Mar. 23, 2022
(86) PCT No.: PCT/EP2022/057620

§ 371 Date: Sep. 19, 2023

(30) Foreign Application Priority Data
Mar. 29, 2021 (GB) 2104417.7

(51) Int. Cl. C07C 251/20; C07C 251/08

(52) U.S. Cl. CPC C07C 251/20; C07C 251/08

(57) ABSTRACT

A compound:

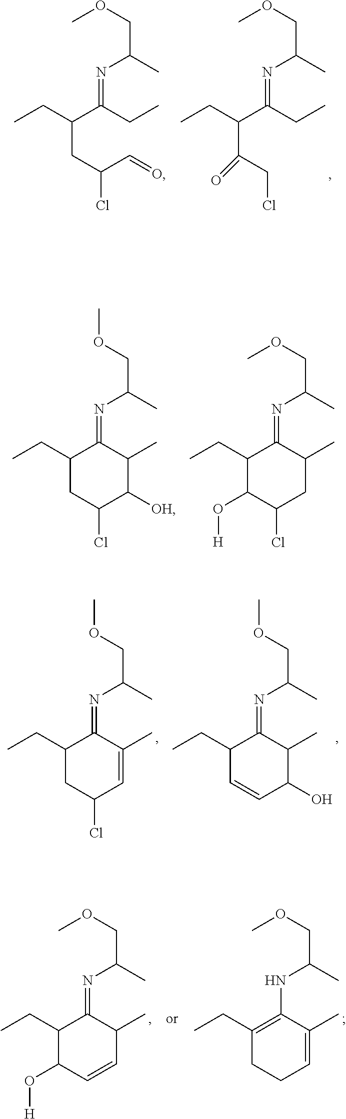

and double bond regioisomers thereof.

The present technology relates to novel intermediates, and isomers thereof.

S-Metolachlor (S-MOC) and metolachlor are part of the chloroacetanilide family of herbicides, used to control grasses and broad-leafed weeds in maize. S-Metolachlor and metolachlor are known to be produced by reacting (S)-NAA or NAA and with chloroacetyl chloride.

The present technology is useful in the production of chemical herbicides S-Metolachlor (S-MOC) and metolachlor. The present invention therefore defines a compound, or double bond regioisomers thereof, as set out in Table 1 below:

TABLE 1

| Compound | Structure |
|---|---|
| 1 | 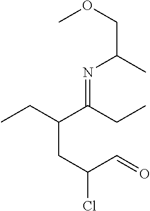 [SMILES: CC/C(=N\C(C)COC)C(CC)CC(Cl)C=O] |
| 2 | 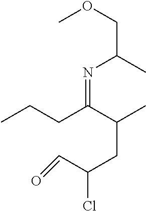 [SMILES: CCC/C(=N/C(C)COC)C(C)CC(Cl)C=O] |
| 3 | 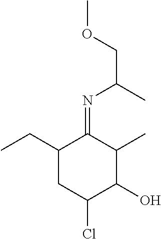 [SMILES: CCC1CC(Cl)C(O)C(C)/C1=N\C(C)COC] |
| 4 | 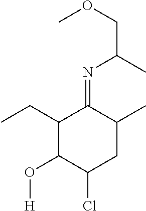 [SMILES: CCC1/C(=N/C(C)COC)C(C)CC(Cl)C1O] |
| 5 | 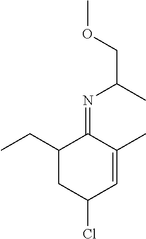 [SMILES: CCC1CC(Cl)C=C(C)/C1=N\C(C)COC] |
| 6 | 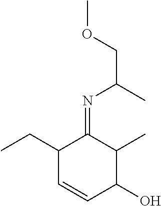 [SMILES: CCC1C=CC(O)C(C)/C1=N\C(C)COC] |
| 7 | 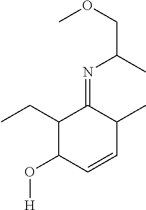 [SMILES: CCC1/C(=N/C(C)COC)C(C)C=CC1O] |
| 8 | 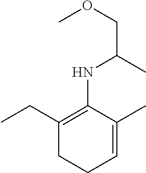 |

The present invention also defines a compound, or double bond regioisomers thereof, as set out in Table 2 below:

TABLE 2

| Compound | Structure |
|---|---|
| 1a | 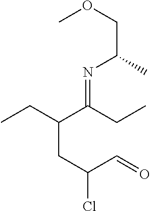 [SMILES: CC/C(=N\[C@@H](C)COC)C(CC)CC(Cl)C=O] |
| 2a | 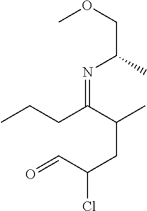 [SMILES: CCC/C(=N/[C@@H](C)COC)C(C)CC(Cl)C=O] |
| 3a | 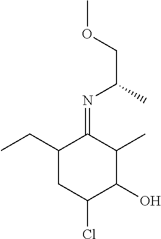 [SMILES: CCC1CC(Cl)C(O)C(C)/C1=N\[C@@H](C)COC] |
| 4a | 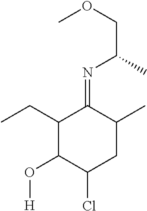 [SMILES: CCC1/C(=N/[C@@H](C)COC)C(C)CC(Cl)C1O] |
| 5a | 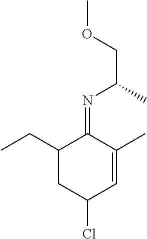 [SMILES: CCC1CC(Cl)C=C(C)/C1=N\[C@@H](C)COC] |
| 6a | 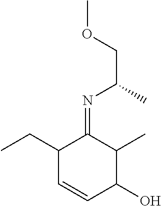 [SMILES: CCC1C=CC(O)C(C)/C1=N\[C@@H](C)COC] |
| 7a | 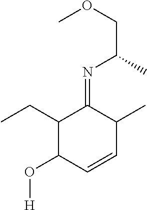 [SMILES: CCC1/C(=N/[C@@H](C)COC)C(C)C=CC1O] |
| 8a | 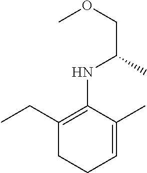 |

Production of (S)-NAA using the novel imine of the present technology is shown below in Schemes 1 through 4:

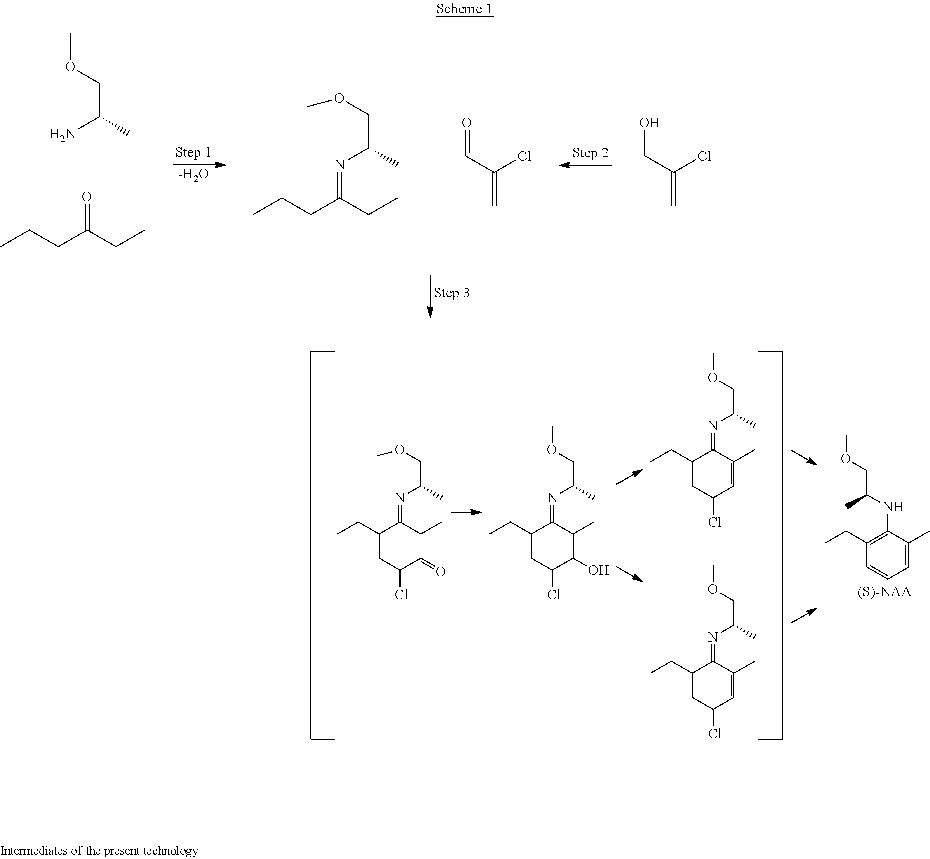

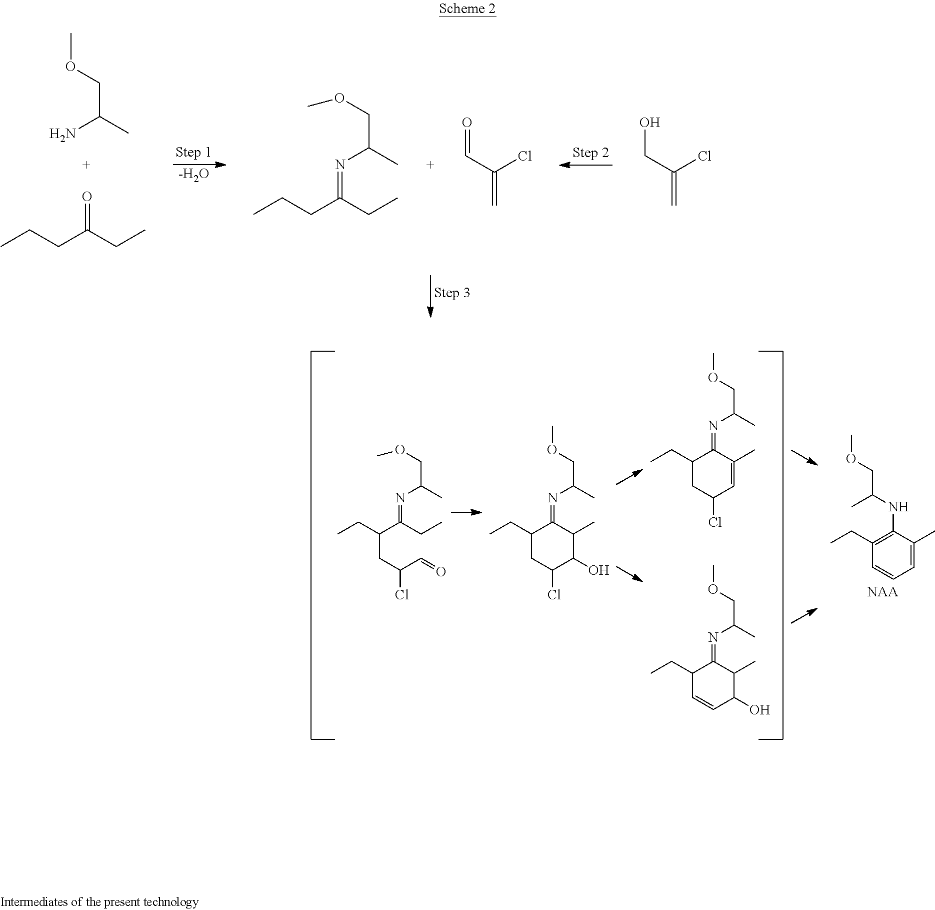

In the case of Schemes 1 and 2, the reaction may proceed to form the alternate regioisomers, i.e., compounds 2, 4 and 7 or compounds 2a, 4a and 7a as defined above.

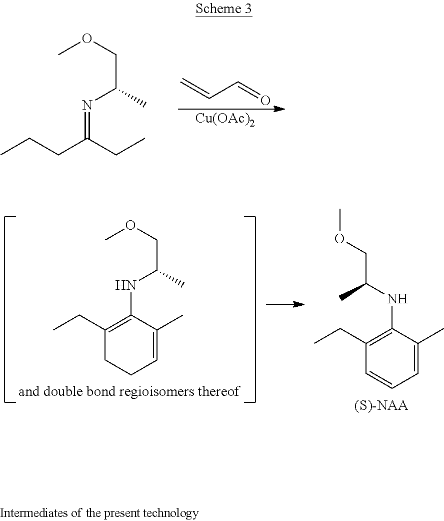

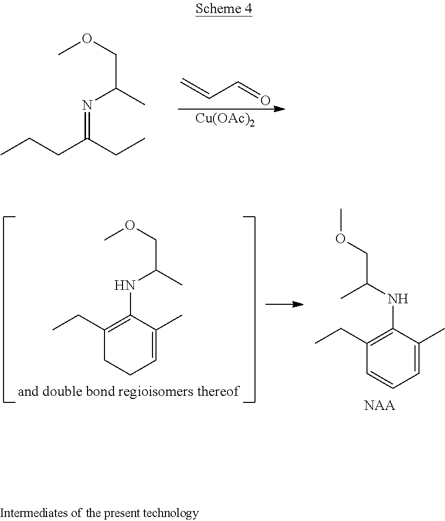

In an alternative embodiment, the reaction may proceed from the acrolein dimethylacetal. This is shown in Scheme 5 (and similarly applies to the racemic mixture):

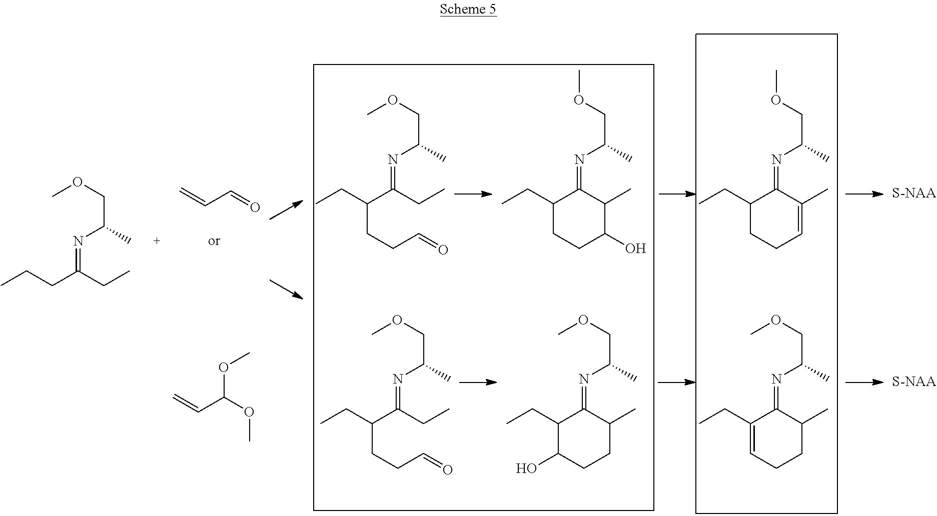

The present invention therefore also defines compounds as set out in Table 3:

TABLE 3

| Compound | Structure |
|---|---|
| 9 | 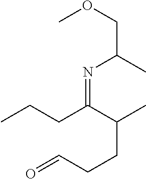 [SMILES: CCC/C(=N/C(C)COC)C(C)CCC=O] |
| 9a | 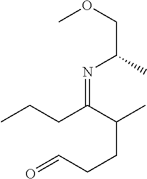 [SMILES: CCC/C(=N/[C@@H](C)COC)C(C)CCC=O] |
| 10 | 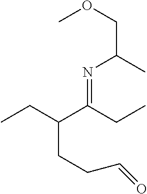 [SMILES: CC/C(=N\C(C)COC)C(CC)CCC=O] |
| 10a | 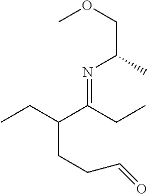 [SMILES: CC/C(=N\[C@@H](C)COC)C(CC)CCC=O] |
| 11 | 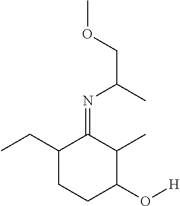 [SMILES: CCC1CCC(O)C(C)/C1=N\C(C)COC] |
| 11a | 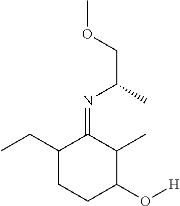 [SMILES: CCC1CCC(O)C(C)/C1=N\[C@@H](C)COC] |
| 12 | 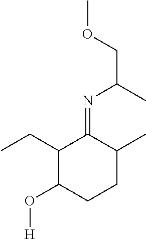 [SMILES: CCC1/C(=N/C(C)COC)C(C)CCC1O] |
| 12a | 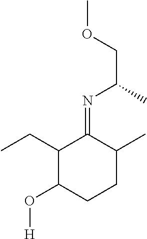 [SMILES: CCC1/C(=N/[C@@H](C)COC)C(C)CCC1O] |

The invention is described by the following non-limiting Examples.

### EXAMPLES

### Experiment 1

The intermediates of the present technology were created during the production of NAA and S-(NAA) in the reaction shown below ((S)-NAA shown):

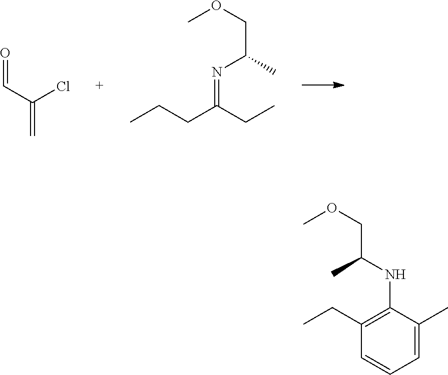

An oven-dried round bottom flask was equipped with nitrogen inlet and magnetic follower. 1,3,5-trimethoxybenzene (10 mol %) charged as internal standard.

Activated molecular sieves (5% w/v) were charged to the reactor. Imine (15 mmol) was added to anhydrous toluene (dried over molecular sieves, 20 mL), mixed and the solution then syringed into the reactor. Chloroacrolein (15 mmol, 1.0 eq) was added to anhydrous toluene and the solution syringed into the reactor. Acetic acid (1.0 eq) was added dropwise over ca. 30 seconds and the reaction then heated to 95° C.(external temperature) on a Drysyn heating block and held with stirring for 3 hr.

The reaction was cooled to room temperature and concentrated under vacuum (35 mbar, 30° C.). Analysis by GC and ¹H NMR showed the desired product S-NAA [S-2-Ethyl—N-(2-methoxy-1-methyl-ethyl)-6-methyl-aniline] had been formed with >99% e.r, in 12% yield (strength 9%).

### Experiment 2

The intermediates of the present technology were created during the production of NAA and S-(NAA) in the reaction shown below ((S)-NAA shown):

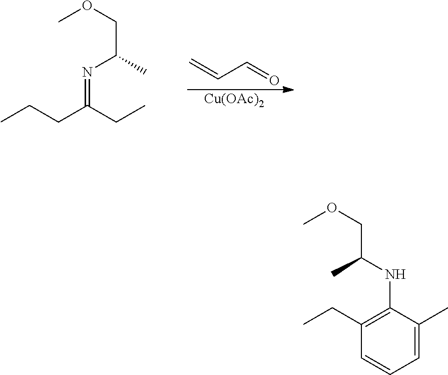

A 100 mL round bottomed flask was equipped with magnetic follower and condenser. To this was added (S)—N-(2-Methoxy-1-methyl-ethyl)hexan-3-imine) (227.6 mg, 1.33 mmol, 1 eq) as a solution in Toluene (6 mL), followed by acrolein (74.5 mg, 88.9 uL,1.33 mmol, 1 eq). Cu(OAc)₂(265.5 mg, 1.33 mmol, 1 eq) was added in one portion, followed by an additional 6 mL of toluene. The mixture was warmed to ˜25° C. internal temperature before the dropwise introduction of trifluoroacetic acid (151.7 mg, 102.9 uL, 1.33 mmol, 1 eq). The reaction was then heated to 110° C. (internal temperature) (˜140° C. heating block temperature) maintained at this temperature for 3 hours.

The reaction mixture was allowed to cool to ambient, then concentrated in vacuo. The product (S)-NAA [ S-2-Ethyl—N-(2-methoxy-1-methyl-ethyl)-6-methyl-aniline] was formed in 60% conversion, and chiral analysis showed desired S-NAA product to have been formed with >99% e.r.

### Experiment 3

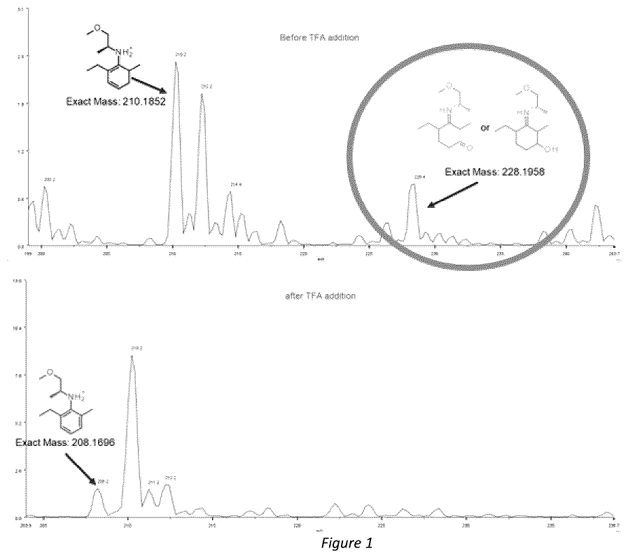

FIG. 1 shows MS spectra demonstrating the formation of compounds 10(a) and/or 11(a) as defined above proceeding according to the scheme:

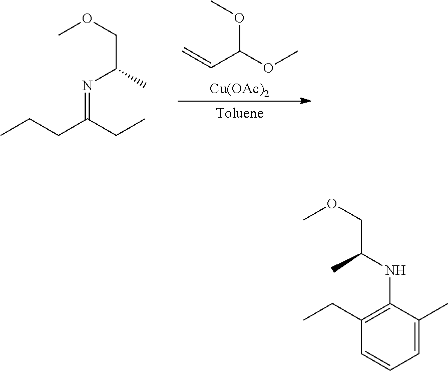

A three-neck round bottomed flask was equipped with magnetic stirrer, condenser and peristaltic pump which allows small samples to be taken and, after dilution in acetonitrile/0.1% formic acid, analysed by mass spectrometry.
(a) (S)—N-(2-Methoxy-1-methyl-ethyl)hexan-3-imine (485 mg, 93% strength, 2.6 mmol) was diluted in toluene (10 ml) and charged into the reactor and stirring started. After ca. 10 minutes, acrolein dimethyl acetal (269 mg, 2.6 mmol) was charged to the vessel as a solution in toluene (6 ml), and conditions held for ca. 8 minutes. Cu(OAc)₂ (525 mg, 2.9 mmol) was charged to the vessel followed by TFA (0.2 mL, 2.6 mmol) and heating initiated. The reaction was then held at reflux for 3 h. Analysis by mass spectroscopy gave spectral data consistent with diene compound 8a.
(b) (S)—N-(2-Methoxy-1-methyl-ethyl)hexan-3-imine (485 mg, 93% strength, 2.6 mmol) was diluted in toluene (16 ml) and charged into the reactor. Molecular sieves (5g) were added, and the mixture heated to reflux with stirring. After 10-20 minutes, acrolein dimethyl acetal (269 mg, 2.6 mmol) in toluene was charged to the vessel toluene, and the mixture held for 30-80 minutes. TFA (0.2 mL, 2.6 mmol) was charged and the reaction held for 40 minutes. Analysis by mass spectroscopy gave spectral data consistent with the alcohol or aldehyde compounds 9a, 10a, 11a, 12a and the diene compound 8a.

The invention is defined by the claims:

1. A compound: 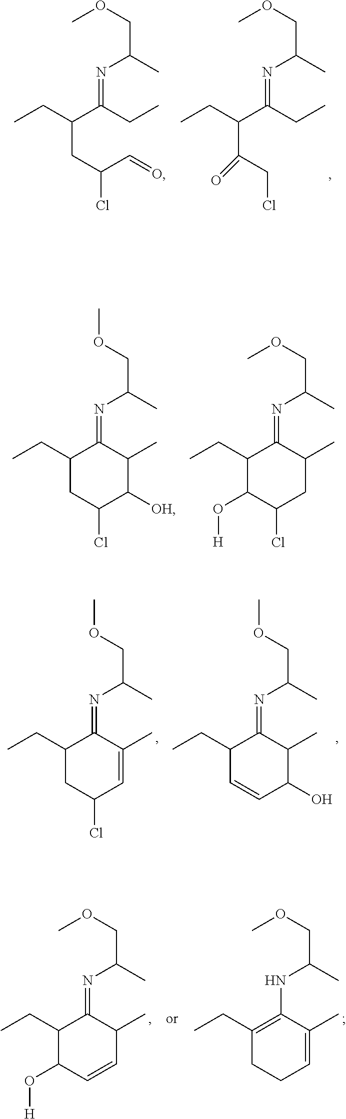
and double bond regioisomers thereof.

2. A compound according to claim 1 selected from: 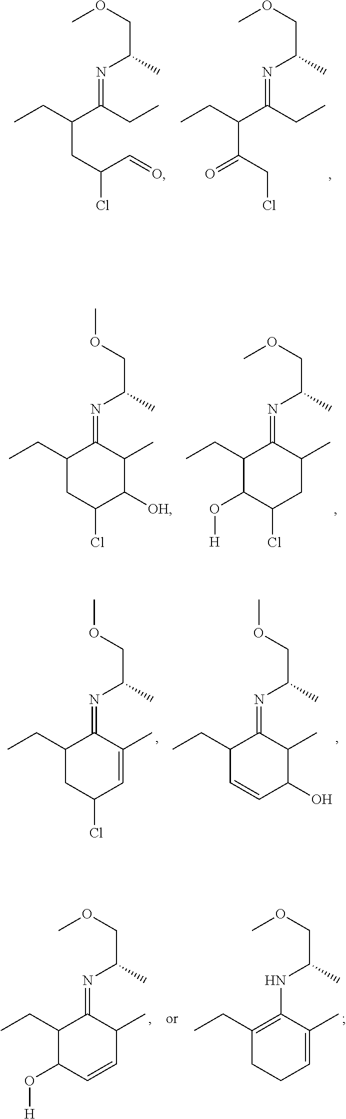
and double bond regioisomers thereof.

3. A compound selected from: [chemical structure]
